(19) United States
(12) Patent Application Publication
(10) Pub. No.: US 2021/0338165 A1
(43) Pub. Date: Nov. 4, 2021

(54) METHOD OF MANAGING DISEASE, AND APPARATUSES OPERATING THE SAME

(71) Applicant: ULIKEKOREA CO., INC., Seoul (KR)

(72) Inventor: Hee Jin KIM, Seoul (KR)

(21) Appl. No.: 17/242,688

(22) Filed: Apr. 28, 2021

Related U.S. Application Data
(63) Continuation-in-part of application No. 15/565,844, filed on Oct. 11, 2017, now abandoned.

(30) Foreign Application Priority Data
Nov. 10, 2016 (KR) 10-2016-0149744
Jun. 10, 2020 (KR) 10-2020-0070584

(51) Int. Cl. A61B 5/00; A61B 5/07; A61B 5/01; A61B 5/11; A61D 17/00

(52) U.S. Cl. CPC A61B 5/6861; A61B 5/073; A61B 5/6871; A61B 5/7275; A61B 5/01; A61B 5/11; A61B 2560/0462; A61D 17/006; A61D 17/008; A61D 17/002; A61B 2503/40; A61B 2562/0219; A61B 2562/162; A61B 5/0008

(57) ABSTRACT

A method of managing a disease and an apparatus performing the same are provided. The method of managing livestock disease includes receiving biometric information of livestock from a biosensor capsule settled in a body of the livestock, detecting a singular point deviating from a reference value, based on the biometric information, monitoring a health state by increasing a disease probability of the livestock when the singular point is detected, and detecting a disease by determining an abnormality in the health state of the livestock when the disease probability of the livestock is a first threshold value or more. An animal subject suspected of reproductive disorders (ovarian cyst) may be rapidly detected, and intensive care (veterinary examination and hormone prescription) may be provided to the farm, thereby increasing productivity of the farm.

### CROSS REFERENCE TO RELATED APPLICATION

This application is a Continuation-In-Part (CIP) of U.S. patent application Ser. No. 15/565,844, filed on Oct. 11, 2017, which is a National Stage filing under 35 U.S.C § 371 of PCT Application No. PCT/KR2016/013616 filed on Nov. 24, 2016 which is based upon and claims the benefit of priority to Korean Patent Application No. 10-2016-0149744 filed on Nov. 10, 2016 in the Korean Intellectual Property Office. This application is also based upon and claims the benefit of priority to Korean Patent Application No. 10-2020-0070584 filed on Jun. 10, 2020 in the Korean Intellectual Property Office. The disclosures of the above-listed applications are hereby incorporated by reference herein in their entireties.

### TECHNICAL FIELD

The following description relates to a method of managing a disease, and apparatuses performing the method.

### BACKGROUND ART

The livestock industry has grown significantly in terms of quantity due to an increase in breeding scale and number of animals due to changes in diet. In detail, with the change from small-scale livestock industry to large-scale livestock industry, farmhouses breeding thousands or tens of thousands of livestock have been created, and as the scale of livestock has increased, a need for a system that may manage livestock in a more systematic manner than before has been required.

As the breeding scale increases, in case of outbreaks of livestock infectious diseases such as foot-and-mouth disease, mad cow disease, swine fever, or avian influenza (AI), there is a problem in which transmission easily occurs between livestock raised in a limited space. In severe cases, large-scale mortality is required, which is a major blow to the livestock industry.

A pen generally refers to a structure built to raise livestock including chickens, ducks, pigs, and cattle, and facilitate the growth and management of livestock.

In such a pen, a large number of livestock live in herds. However, in case of an outbreak of an infectious and transmissible disease, such a group living may lead to extensive damage because all animals in a pen may be infected with the disease.

For instance, a huge number of cattle may die from some fatal, infectious diseases such as bovine spongiform encephalopathy (BSE), which is casually referred to as a mad-cow disease, each year causing heavy losses to stock farms.

### DISCLOSURE

### Technical Goals

An aspect of the present disclosure provides technology for managing and controlling a bovine disease.

Another aspect of the present disclosure also provides technology for readily and conveniently providing a biosensor capsule that is orally administered directly to cattle without a need for an expensive gathering box and transmits bioinformation to determine a health state of the cattle.

### Technical Solutions

According to an aspect of the present disclosure, a method of managing livestock disease includes receiving bioinformation of livestock from a biosensor capsule settled in a body of the livestock; detecting a singular point deviating from a reference value, based on the bioinformation; monitoring a health state by increasing a disease probability of the livestock when the singular point is detected; and detecting a disease by determining an abnormality in the health state of the livestock when the disease probability of the livestock is a first threshold value or more.

The bioinformation may include temperature information of the livestock and activity information of the livestock.

The activity information of the livestock may include at least one of angular velocity information and acceleration information sensed by the biosensor capsule.

The detecting of the singular point may include an estrus detection operation of determining estrus when at least one of a first singular point in which the temperature information exceeds a temperature threshold value or a second singular point in which the activity information exceeds an activity threshold value is expressed.

When the estrus is detected, a range of an increase in the disease probability of the livestock may be greater.

In the estrus detection operation, when a duration of the first singular point or a duration of the second singular point lasts a first duration or more, it may be determined as the estrus.

In the estrus detection operation, when both the first singular point and the second singular point are expressed, it may be determined as the estrus.

In the estrus detection operation, when only one of the first singular point and the second singular point is expressed and a duration of an expressed singular point lasts a second duration or more, it may be determined as the estrus.

In the monitoring of the health state, a range of change in the disease probability of the livestock may be determined according to a frequency of expression of the estrus, an irregularity of expression of the estrus, a duration of expression of the estrus, and intensity of expression of the estrus.

In the monitoring of the health state, as the frequency of expression of the estrus increases, the expression of the estrus is irregular and the duration of the estrus increases, a range of an increase in the disease probability of the livestock may be controlled to greater.

When the estrus detection operation is expressed three times or four times or more within a second estrus period, the disease probability of the livestock may be controlled to be equal to or higher than the first threshold value.

In the monitoring, the disease probability of the livestock may be lowered after the detecting of the singular point and before detection of a next singular point.

The method may further include an alarm operation of notifying a user of a disease of the livestock when the disease probability is equal to or higher than a set value.

The abnormality in the health state of the livestock may be an ovarian cyst.

The method may further include inserting and seating the biosensor capsule in a stomach or a vagina of the livestock.

According to an aspect of the present disclosure, a disease management server managing a disease of livestock includes a communication module receiving bioinformation of the livestock from a biosensor capsule settled in a body of the livestock; and a controller analyzing the bioinformation, determining a health state of the livestock based on an analysis result, and transmitting information on the health state of the livestock to a user managing the livestock. The controller detects a singular point deviating from a reference value based on the bioinformation, increases a disease probability of the livestock when the singular point is detected, and transmits an abnormality in the health state of the livestock to the user when the disease probability of the livestock is equal to or higher than a first threshold value.

The bioinformation may include temperature information of the livestock and activity information of the livestock, and the controller may determine estrus when at least one of a first singular point in which the temperature information exceeds a temperature threshold value or a second singular point in which the activity information exceeds an activity threshold value is expressed, and may increase the disease probability of the livestock.

The controller may control a range of a change in the disease probability of the livestock, to be changed depending on a frequency of expression of the estrus, irregularity of expression of the estrus, a duration of expression of the estrus, and intensity of expression of the estrus.

The controller may control a range of a change in the disease probability of the livestock, to be changed depending on a frequency of expression of the estrus, irregularity of expression of the estrus, a duration of expression of the estrus, and intensity of expression of the estrus.

The abnormality in the health state of the livestock may include an ovarian cyst.

As set forth above, according to an example embodiment, a disease management server 300 may quickly detect an animal suspected of having a reproductive disorder (ovarian cyst) using data collected by a biosensor capsule 200, and may provide intensive care for the animal (examination and hormone prescription by a veterinarian) on the farm.

### BRIEF DESCRIPTION OF DRAWINGS

FIG. 1 is a diagram illustrating a disease management system according to an example embodiment.

FIG. 2 is a diagram illustrating a biosensor capsule illustrated in FIG. 1.

FIG. 3 is a diagram illustrating a disease management server illustrated in FIG. 1.

FIGS. 4A through 4D are diagrams illustrating examples of information associated with a health state of a cow that is provided by a disease management server according to an example embodiment.

FIG. 5 is a flowchart illustrating an operating method of a disease management server illustrated in FIG. 1.

FIG. 6 is a diagram illustrating a type of a follicle and an estrus period depending on hormonal changes in livestock.

FIG. 7 is a table illustrating summarized information on experimental animals.

FIG. 8 is a conceptual diagram of a Long Short-Term Memory (LSTM)-Fully Convolutional Networks (FCN) model.

FIG. 9 is graphs illustrating bioinformation obtained from a biosensor capsule of a normal animal.

FIG. 10 is graphs illustrating bioinformation obtained from a biosensor capsule of an experimental animal having an ovarian cyst.

FIGS. 11 to 12 are graphs illustrating an estrus probability and an ovarian cyst probability calculated based on temperature information and activity information of livestock.

FIG. 13 is a table illustrating experimental results of experimental subjects.

### BEST MODE FOR CARRYING OUT INVENTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the present disclosure, with the exception of operations necessarily occurring in a certain order. Also, descriptions of features that are known in the art may be omitted for increased clarity and conciseness.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

Terms such as first, second, A, B, (a), (b), and the like may be used herein to describe components. Each of these terminologies is not used to define an essence, order, or sequence of a corresponding component but used merely to distinguish the corresponding component from other component(s). For example, a first component may be referred to as a second component, and similarly the second component may also be referred to as the first component.

It should be noted that if it is described in the specification that one component is “connected,” “coupled,” or “joined” to another component, a third component may be “connected,” “coupled,” and “joined” between the first and second components, although the first component may be directly connected, coupled or joined to the second component. In addition, it should be noted that if it is described in the specification that one component is “directly connected” or “directly joined” to another component, a third component may not be present therebetween. Likewise, expressions, for example, “between” and “immediately between” and “adjacent to” and “immediately adjacent to” may also be construed as described in the foregoing.

The terminology used herein is for the purpose of describing particular embodiments only and is not intended to be limiting. As used herein, the singular forms “a,” “an,” and “the,” are intended to include the plural forms as well, unless the context clearly indicates otherwise. It will be further understood that the terms “comprises,” “comprising,” “includes,” and/or “including,” when used herein, specify the presence of stated features, integers, operations, elements, and/or components, but do not preclude the presence or addition of one or more other features, integers, operations, elements, components, and/or groups thereof.

Unless otherwise defined, all terms, including technical and scientific terms, used herein have the same meaning as commonly understood by one of ordinary skill in the art to which this disclosure pertains based on an understanding of the present disclosure. Terms, such as those defined in commonly used dictionaries, are to be interpreted as having a meaning that is consistent with their meaning in the context of the relevant art and the present disclosure, and are not to be interpreted in an idealized or overly formal sense unless expressly so defined herein.

Hereinafter, some example embodiments will be described in detail with reference to the accompanying drawings. Regarding the reference numerals assigned to the elements in the drawings, it should be noted that the same elements will be designated by the same reference numerals, wherever possible, even though they are shown in different drawings.

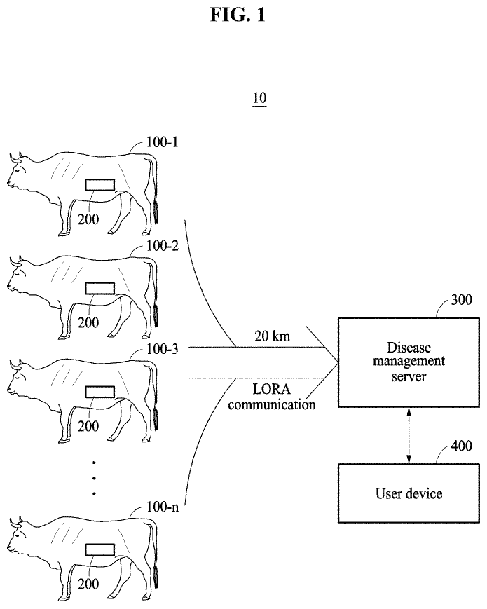

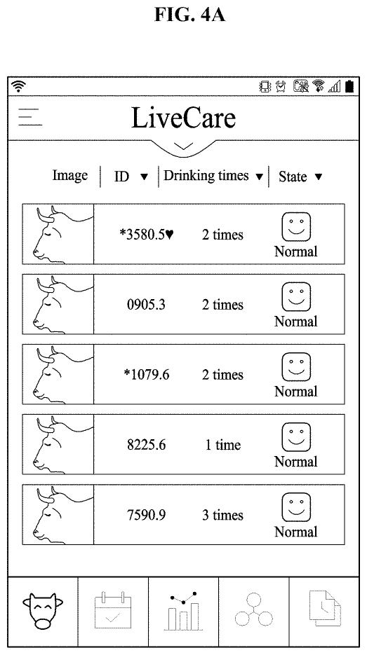

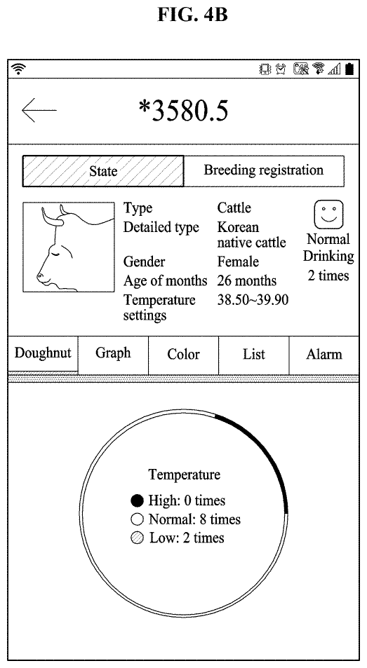

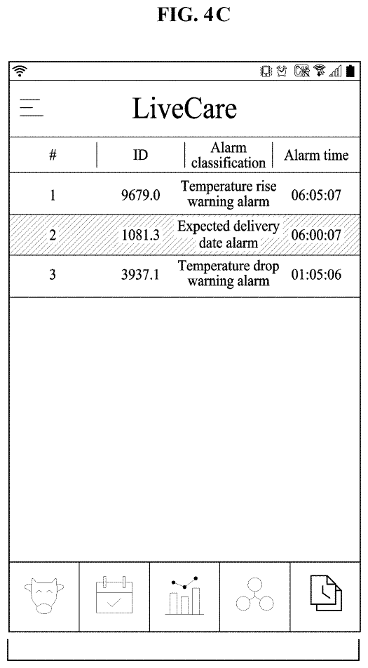

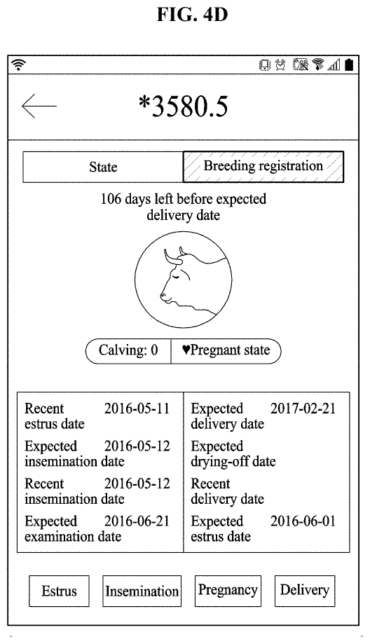

FIG. 1 is a diagram illustrating a disease management system according to an example embodiment. FIGS. 4A through 4D are diagrams illustrating examples of information associated with a health state of a cow that is provided by a disease management server according to an example embodiment.

Referring to FIG. 1, a disease management system 10 includes a plurality of cows, for example, a cow 100-1 through a cow 100-n in which n denotes a natural number greater than 1, a disease management server 300, and a user device 400.

A biosensor capsule 200 is provided in each of the cows 100-1 through 100-n. The cows 100-1 through 100-n may be managed by a user of the user device 400. In addition, some of the cows 100-1 through 100-n may be managed by another user different from the user of the user device 400.

An identification (ID) may be assigned to each of the cows 100-1 through 100-n to be managed by the disease management server 300. For example, the cows 100-1 through 100-n may be classified by each ID assigned to the biosensor capsule 200, and managed by the disease management server 300 based on the assigned ID.

The cows 100-1 through 100-n may transmit bioinformation to the disease management server 300 through the biosensor capsule 200. The biosensor capsule 200 may be placed and settled, or provided (used hereinafter), at a predetermined location in a body of each of the cows 100-1 through 100-n. Thus, the biosensor capsule 200 may obtain the bioinformation from the cows 100-1 through 100-n and transmit the obtained bioinformation to the disease management server 300.

The disease management server 300 may manage the cows 100-1 through 100-n, and provide the user device 400 with a service to remotely manage the cows 100-1 through 100-n. For example, the disease management server 300 may provide the service for management of the cows 100-1 through 100-n based on the bioinformation obtained from the biosensor capsule 200 of the cows 100-1 through 100-n.

The disease management server 300 may analyze the bioinformation, determine respective health states of the cows 100-1 through 100-n based on a result of the analyzing, and transmit the determined health states to the user device 400. Here, a health state of a cow may include at least one of a breeding-related state associated with estrus and delivery of the cow, a disease state of the cow, a methane gas generation state of the cow, or a rumen activity state of the cow.

In addition, the disease management server 300 may provide the user device 400 with an alarm that is classified based on a health state of a cow.

A disease management service application that may implement the service provided by the disease management server 300 may be installed and implemented in the user device 400, and perform a function associated with a disease management service. For example, the disease management service application may be downloaded from the disease management server 300 to be installed. For another example, the disease management service application may be downloaded from an application store or an Android market to be installed.

The user device 400 may receive, from the disease management server 300, the health states of the cows 100-1 through 100-n and/or the alarm that is classified based on a health state of a cow or corresponds to a health state of a cow. Here, information associated with a health state of a cow may include, for example, information associated with estrus, insemination, pregnancy, and delivery of the cow. The breeding-related state may include information associated with a recent estrus date, an expected insemination date, a recent insemination date, an expected examination date, an expected delivery date, an expected drying-off date, a recent delivery date, and an expected estrus date.

Examples of the information associated with the health state that is provided by the disease management server 300 to the user device 400 are illustrated in FIGS. 4A through 4D.

The user device 400 may be embodied as a personal computer (PC), a data server, or a portable electronic device.

The portable electronic device may be embodied as a laptop computer, a mobile phone, a smartphone, a tablet PC, a mobile Internet device (MID), a personal digital assistant (PDA), an enterprise digital assistant (EDA), a distal still camera, a digital video camera, a portable multimedia player (PMP), a personal or portable navigation device (PND), a handheld game console, an e-book, or a smart device. The smart device may be embodied as a smart watch, a smart band, or a smart ring.

As described, the user may remotely manage a plurality of objects, for example, the cows 100-1 through 100-n as illustrated, collectively through the user device 400 and continuously manage a history of each object, and receive information on a real-time state of each object and receive an alarm corresponding to a health state of each object to respond rapidly and accordingly.

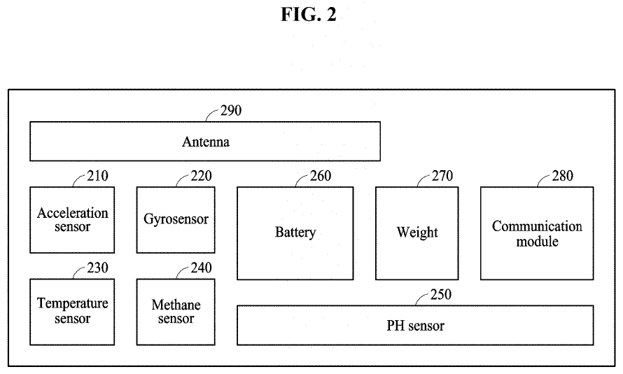

FIG. 2 is a diagram illustrating the biosensor capsule 200 illustrated in FIG. 1.

Referring to FIG. 2, the biosensor capsule 200 includes a plurality of sensors, for example, an acceleration sensor 210, a gyrosensor 220, a temperature sensor 230, a methane sensor 240, and a potential hydrogen (pH) sensor 250, a battery 260, a weight 270, a communication module 280, and an antenna 290.

The biosensor capsule 200 may be injected into and settled at a predetermined location in the body of the cow, for example, may enter the stomach and be seated in the rumen or be seated in the vagina. In this case, the biosensor capsule 200 may be protected by a case (not illustrated) used for protection from moisture and gastric acid in the body of the cow.

The acceleration sensor 210 may obtain acceleration information of the cow. For example, the acceleration sensor 210 may sense, in real time, an acceleration value of the cow and generate the acceleration information.

The biosensor capsule 200 may change orientation while being in a livestock body, for example, in the rumen and/or in the reticulum of a cow. Therefore, detecting livestock's movement and/or gastrointestinal activity in only one or both axes may result in inaccurate calculations.

Therefore, the acceleration sensor 210 may be a 3-axis or 6-axis accelerometer capable of detecting animal's movement and/or gastrointestinal activity on each of “x”, “y” and “z” axes of Cartesian coordinates. As the number of detection axes increases, a motion may be detected more accurately. For example, when a 6-axis acceleration sensor is used, more precision may be obtained compared to measuring activity with a 3-axis acceleration sensor.

An acceleration vector magnitude (v) may be calculated from a value measured with the 3-axis accelerometer by calculating a square root of a sum of squares of each of the “x”, “y” and “Z” coordinate axes.

More detailed information about an actual movement of livestock may be obtained by differentiating the vector magnitude to remove an error rate generated by a movement caused by shaking of the biosensor capsule 200 itself in the body of the livestock or other acceleration force, for example, gravity, acting on the livestock.

In addition to detecting the movement and/or gastrointestinal activity of livestock, an accelerometer 221 may be configured to detect gastrointestinal contractions. Since the biosensor capsule 200 may be placed in the stomach, e.g., in the rumen or the reticulum of ruminant livestock, the biosensor capsule 200 may be affected such as movement or the like due to the gastrointestinal contractions of livestock. Therefore, the biosensor capsule 200 may be configured to have a sufficient weight and/or density to be maintained in a lower part of the livestock stomach and/or to be moved to and located on a stomach wall of the livestock.

The biosensor capsule 200 coupled to move along with the stomach of livestock is configured to match and correct the movement of the biosensor capsule 200 corresponding to the movement of the stomach in such a manner that the accelerometer 221 of the biosensor capsule 200 may detect a motion of the stomach such as contractions, thereby referring to values thereof in extracting a movement value of the stomach.

For example, typical ruminant livestock, under normal health, may make gastrointestinal contractions three times per minute for a rumination activity period of time, for example, for 20 seconds each time.

Non-rumination time may indicate the health state of livestock in which the livestock no longer feeds and/or feeds intermittently.

Thus, a manager may detect when livestock becomes “non-rumination” by observing changes and/or decreases in the frequency of gastrointestinal contractions of the livestock.

An increase in non-rumination time of the livestock may indicate a serious health state.

In addition, the livestock may exhibit “non-rumination” before unusual health-state symptoms, e.g., increased/decreased movement or temperature, or the like, appear. The gyrosensor 220 may obtain angular speed information of the cow. For example, the gyrosensor 220 may sense, in real time, an angular speed value of the cow and generate the angular speed information.

Based on the principle of a gyroscope, MEMS technology may be applied to implement a gyrosensor in the form of a chip, and the gyrosensor may be mounted in the biosensor capsule 200 due to a relatively small size thereof.

Using the acceleration sensor 210 and the gyrosensor 220, movement of the biosensor capsule 200 may be sensed, and as a result, the activity of the cow may be sensed. A roll, pitch, and yaw of the biosensor capsule may be detected by combining an acceleration detected by the acceleration sensor and an angular velocity detected by the gyrosensor.

A temperature sensor 230 may obtain temperature information in the stomach or vagina of the cow. For example, the temperature sensor 230 may detect a temperature value of a cow's stomach in real time and generate temperature information. The temperature sensor 230 may include a thermistor, a thermocouple, a platinum resistance thermometer, or the like. The gastric or vaginal temperature of the cow and the body temperature of the cow may have a constant difference in values thereof.

The methane sensor 240 may obtain methane gas generation information of the stomach of the cow. For example, the methane sensor 240 may sense, in real time, an amount of methane gas generated in the stomach of the cow and generate the methane gas generation information.

The pH sensor 250 may obtain pH information of the stomach of the cow. For example, the pH sensor 250 may sense, in real time, a pH level of the stomach of the cow and generate the pH information.

An electrical conductivity sensor is a sensor that measures electrical conductivity of a substance present in the stomach or vagina. Electrical conductivity is an index indicating the degree to which current flows well in substances or objects located between a pair of sensor electrodes. A conductor such as a metal has relatively high electrical conductivity, while a nonconductor such as glass has low electrical conductivity.

Although electrical conductivity between two points of a solid such as a metal may be measured, electrical conductivity may also be measured in a liquid containing electrolyte ions. The electrical conductivity of a liquid is further increased as the concentration of the electrolyte ions contained in the liquid increases, and the unit thereof is dS/m or mS/cm, and the electrical conductivity may vary depending on pH concentration and temperature. The electrical conductivity slightly changes depending on the type of ions contained in the liquid, but may change in proportion to a total salt concentration.

The acceleration information, the angular speed information, the temperature information, the methane gas generation information, and the pH information may be transmitted to the disease management server 300 through the communication module 280 and the antenna 290.

The battery 260 may supply power to components of the biosensor capsule 200, for example, the acceleration sensor 210, the gyrosensor 220, the temperature sensor 230, the methane sensor 240, the pH sensor 250, the communication module 280, and the antenna 290. For example, the battery 260 may be embodied in various types of battery.

The battery 260 may include a battery power storage device such as a lithium ion battery, a lead battery, a nickel cadmium battery, or the like.

In another embodiment, the battery 260 may include a generator. The generator may include a piezoelectric generator or a mass/alternating current (AC) power source generator that generates power from movement and/or kinetic activity or vibrations of the biosensor capsule 200 in the host animal.

In another example embodiment, the generator may be a heat-activated generator for generating electrical energy from a body heat of the host animal. The generator may include a microbial fuel cell (MFC) configured to generate electrical energy derived from bacteria that are fed to organic matter in the stomach of the livestock, e.g., rumen or reticulum.

The generator may be disposed outside of a biosensor capsule housing. The battery 260 may include both the battery power storage device and the generator. In this embodiment, power generated by the generator 264 may be stored in the battery power storage device.

The battery 260 may include the battery power storage device and/or a power monitor that may be used to monitor power and/or voltage levels of the generator. Multiple power-save operations may be performed using power state information provided by a power monitor 266.

The weight 270 may be provided to add a weight so that the biosensor capsule 200 is injected into the body of the cow not to be externally discharged and to be settled at a predetermined location, for example, the stomach, in the body of the cow. That is, the weight 270 may have a sufficient weight to allow the biosensor capsule 200 not to be externally discharged, but to be settled in the stomach of the cow, during a ruminating process of the cow.

Hereinafter, how the biosensor capsule 200 is not externally discharged by the weight 270 will be described as follows.

A cow is a ruminant animal that plucks grass in a rush and places the grass into a stomach without chewing the grass, and ruminates on the grass or food in a safe place, because the cow, an herbivore, is anxious about a potential attack by a carnivore.

A stomach of a ruminant animal is divided into four stomachs, as also shown in a case of a cow. The four stomachs includes a first stomach or a paunch that stores food eaten in a rush and dominates approximately 80% of a total stomach volume of a cow, a second stomach, or a reticulum or a honeycomb, that mixes the food and spits the food back to a mouth of the cow, and a third stomach, or a psalterium or a manyplies, and a fourth stomach, or an abomasum or a read, through which the food ruminated on in the mouth moves to intestines of the cow.

For example, food that is swallowed without being chewed by a cow becomes a lump to enter the first stomach or the paunch that is the largest of the four stomachs. The food becomes more wet and softer through the first stomach, and enters the second stomach or the reticulum. The food is divided into suitable size portions in the second stomach. The food is brought back from the second stomach to a mouth of the cow, and is ruminated on when the cow is free. Thus, the cow chews over and over all day. When the cow swallows again the food after chewing, the food moves to the third stomach or the psalterium and the fourth stomach or the abomasum, and finally to intestines of the cow. Through such a process, the cow digests the food.

Such a complex digestion process may take more than three days, and the cow may consume all nutrients contained in the food in the digestion process. In general, a healthy cow may start ruminating on a feed 30 to 40 minutes after the cow eats the feed, and the ruminating may be repeated six to eight times for 40 to 50 minutes. Here, the feed of approximately 50 to 60 kilograms (kg) may be ruminated on. That is, the cow may eat for eight hours, ruminate for eight hours, and rest for eight hours per day.

The biosensor capsule 200 may be injected into the body of the cow, and then be settled at a predetermined location, for example, the first stomach or the paunch, in the body of the cow. The first stomach or the paunch may dominate 80% of a total volume of the stomach of the cow, and include a plurality of spaces. Thus, the first stomach or the paunch may have a suitable space in which the biosensor capsule 200 is to be settled.

As described, a cow, which is a ruminant animal that ruminates on food, may ruminate on a considerable amount of food brought back to a mouth of the cow from the first stomach (paunch) and the second stomach (reticulum). In such a ruminating process, the biosensor capsule 200 may be mixed with the food and then discharged again to a mouth of the cow.

In addition, the first stomach or the paunch has a plurality of spaces, and food in the stomach of the cow may be mixed with water in general. Thus, when the biosensor capsule 200 is not weighed sufficiently, the biosensor capsule 200 may float above the food due to the water in such a mixing process, and be discharged to the mouth along with the food in the ruminating process.

Thus, using the weight 270, the biosensor capsule 200 may be mixed with food and move along with the food in the spaces of the stomach of the cow, and then sink to a bottom of a space by gravity and move there, and then be naturally settled at a suitable location not to be discharged outside the body and to allow a plurality of sensors, for example, the sensors 210 through 250, of the biosensor capsule 200 to accurately measure internal information of the cow.

The communication module 280 and the antenna 290 may be used to perform communication between the biosensor capsule 200 and the disease management server 300. That is, the biosensor capsule 200 and the disease management server 300 may exchange signals or data through the communication module 280 and the antenna 290.

When the amount of data to be transmitted is relatively small, a communication distance may be much more important for communication of data transmission over long distances rather than for high throughput data transmission. To satisfy both the amount of transmitted data and the transmission distance, battery consumption is relatively great and antenna performance should be good.

By reducing a data rate by 4 times, the transmission distance may be doubled. The biosensor capsule 200 injected into the body of the livestock may be difficult to use a high-power short-wave signal capable of transmitting a signal over a long distance. Therefore, data measured by the biosensor capsule 200 may be transmitted using a relatively low frequency, Sub-1 GHz band.

In order for radio signals to penetrate livestock's body, the use of long waves may be preferred for efficient and medical reasons. For example, for long-distance communication, there may be a problem with a relatively great amount of power consumption, and if the power consumption is great, since the lifespan of the battery is short, the biosensor capsule 200 should be repeatedly removed from and injected into the body of the livestock to replace or charge the battery 130.

The communication module 280 according to an example embodiment of the present disclosure may use a communication method such as Bluetooth, Wi-Fi or the like, and may use BLE, Wi-Fi HaLow, or the like, in detail, designed for low power. The communication module 280 according to an example embodiment of the present disclosure may transmit a signal using a frequency band of Sub-1 GHz.

Even when such a low-frequency signal of 1 GHz or less is used, since radio waves are attenuated by passing through the body of livestock, a transmission distance of a signal capable of transmission of a long distance of 6.4 km may be reduced to about 100 m to several hundreds of m. Accordingly, the communication module 280 may transmit a signal to the disease management server 300 through a repeater located in a livestock house or a separate device within a transmission distance.

In the case in which a wireless repeater is installed far, outside the communication distance from the biosensor capsule 200, it may be difficult to directly transmit data from the biosensor capsule 200 to the disease management server 300 through the wireless repeater. In this case, a second device may be installed on another part of the livestock's body (not illustrated).

The second device may be configured to receive data from the biosensor capsule 200 installed in the livestock's body and transmit the data to the wireless repeater (or a drone (not illustrated)) or a base station. Examples of the second device may include a necklace, an ear-tag (not illustrated), and the like.

The communication module 280 may be embodied as a long-range (LORA) communication module. Thus, without a need for a gathering device between the biosensor capsule 200 and the disease management server 300, the biosensor capsule 200 may transmit the bioinformation to the disease management server 300 by accessing a communication network within a distance of 20 kilometers (km) through the communication module 280.

The sensors 210 through 250 may be integrated in a printed circuit board (PCB). In addition, the communication module 280 and/or the antenna 290 may also be integrated in the PCB.

As described, the biosensor capsule 200 may be orally administered directly to a cow, and be thus installed readily and conveniently without a need for an expensive gathering box.

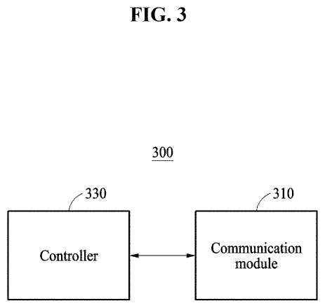

FIG. 3 is a diagram illustrating the disease management server 300 illustrated in FIG. 1.

Referring to FIG. 3, the disease management server 300 includes a communication module 310 and a controller 330.

The communication module 310 may perform communication between the disease management server 300 and the biosensor capsule 200. In addition, the communication module 310 may perform communication between the disease management server 300 and the user device 400. That is, the disease management server 300 and the biosensor capsule 200 may exchange signals or data through the communication module 310, and the disease management server 300 and the user device 400 may exchange signals or data through the communication module 310.

The controller 330 may control an overall operation of the disease management server 300. The controller 330 may receive bioinformation of a cow from the biosensor capsule 200 through the communication module 310.

The controller 330 may analyze the bioinformation, and determine a health state of the cow based on a result of the analyzing. The health state may include at least one of a breeding-related state associated with estrus and delivery of the cow, a disease state of the cow, a methane gas generation state of the cow, or a rumen activity state of the cow.

For example, the controller 330 may determine an estrus state and/or a delivery state of the cow based on angular speed information, acceleration information, and temperature information, which are included in the bioinformation.

For another example, the controller 330 may determine an activity state of a stomach of the cow based on the angular speed information, the acceleration information, and pH information, which are included in the bioinformation.

For still another example, the controller 330 may determine an amount of methane gas generated in the stomach of the cow based on methane gas generation information included in the bioinformation.

For yet another example, the controller 330 may determine the disease state of the cow based on the angular speed information, the acceleration information, the temperature information, a pH level, and the methane gas generation information, which are included in the bioinformation.

The controller 330 may transmit information associated with the health state to the user device 400 through the communication module 310. Here, information associated with the breeding-related state may include information associated with estrus, insemination, pregnancy, and delivery of the cow. In addition, the information associated with the breeding-related state may also include information associated with a recent estrus date, an expected insemination date, a recent insemination date, an expected examination date, an expected delivery date, an expected drying-off date, a recent delivery date, and an expected estrus date of the cow.

The disease management server 300 may provide the user device 400 with an alarm corresponding to the health state, for example, a temperature rise warning alarm, a temperature drop warning alarm, an alarm for the expected delivery date, an alarm for the expected examination date, an alarm for the expected drying-off date, and an alarm for the expected estrus date.

A more detailed operation of the controller 330 will be described later.

The term “module” used herein may indicate hardware that performs functions and operations corresponding to each component designated the same herein, a computer program code that performs or implements certain functions and operations, or an electronic recording medium equipped with a computer program code that performs or implements certain functions and operations, for example, a processor and a microprocessor.

The module may indicate a functional and/or structural combination of hardware to perform technical concepts or features described herein and/or software to implement the hardware.

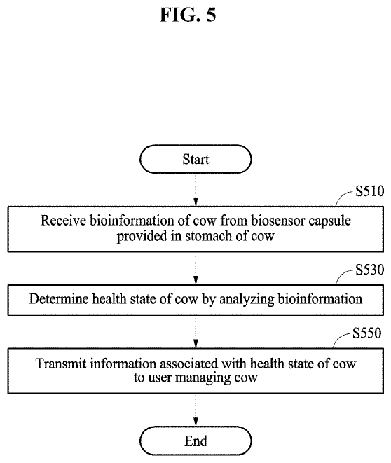

FIG. 5 is a flowchart illustrating an operating method of the disease management server 300 illustrated in FIG. 1.

Referring to FIG. 5, in S510, the controller 330 receives bioinformation of a cow from the biosensor capsule 200 provided in a stomach of the cow through the communication module 310.

In S530, the controller 330 determines a health state of the cow by analyzing the bioinformation.

In S550, the controller 330 transmits information associated with the health state of the cow to a user managing the cow through the communication module 310. The health state may include at least one of a breeding-related state associated with estrus and delivery of the cow, a disease state of the cow, a methane gas generation state of the cow, or a rumen activity state of the cow.

In the flowchart of FIG. 5, although the biosensor capsule 200 is illustrated as being settled in the stomach of the cow, the biosensor capsule 200 may be applied to all forms that may be injected into the livestock's body, such as into the vagina.

Among the health states of the livestock that may be detected using the biosensor capsule 200, the reproductive disorders of livestock may be detected as a representative. A representative example of the cause of the reproductive disorders is an ovarian cyst, and in the case in which the reproductive disorder is detected early, the damage of the reproductive disorder may be significantly reduced, contributing to the productivity of cattle and the economy of farmhouses.

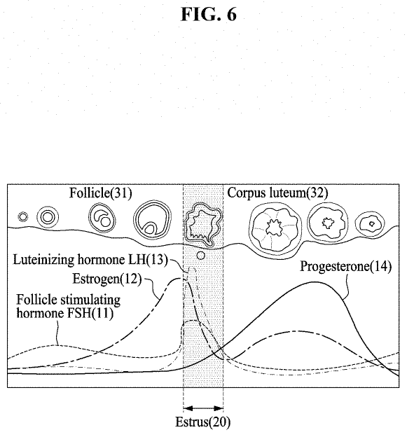

FIG. 6 is a diagram illustrating the type of follicles and estrus time according to hormonal changes in livestock.

In livestock such as pigs and cows, if females do not become pregnant, estrus repeats at regular intervals, and shows an estrus cycle of about 21 days (around 3 weeks), including the follicle phase 4-5 days and the luteal phase 16 days. A follicle 31 in the female ovary is affected by follicle-stimulating hormone (FSH) 11 secreted from the pituitary gland to promote growth and development, and estrogen 12 is secreted from the fully developed follicle 31.

The estrogen 12 affects the hypothalamus or pituitary gland of the brain, and the secreted amount of estrogen 12 increases as the follicle grows. When the estrogen 12 has a predetermined concentration or more, the livestock is introduced into an estrus period 20, and the estrogen 12 affects the hypothalamus and pituitary gland of the brain to induce the secretion of luteinizing hormone (LH) 13 from the pituitary gland. When the luteinizing hormone 13 is secreted, the mature follicle 31 ovulates (discharges an egg into the uterus) and the follicle 31 is converted into the corpus luteum 32. Immediately after the ovulation, the concentration of the estrogen 12 decreases, and a concentration of progesterone 14 secreted from the corpus luteum 32 increases rapidly.

The corpus luteum 32 disappears after about 15 days, and the concentration of the progesterone 14 changes according to the development cycle of the corpus luteum. The progesterone 14 serves to maintain pregnancy by inhibiting uterine contraction and ovulation, and if pregnancy does not occur, the corpus luteum 32 degenerates and the concentration of progesterone 14 decreases.

Ovarian cyst (OC) is a functional disease caused by abnormal secretion of these hormones, and occurs due to decreased secretion of luteinizing hormone (LH) or excessive secretion of follicle stimulating hormone (FSH).

The ovarian cyst (OC) refers to the presence of at least one structure that stores a fluid and has a diameter of 2.5 cm or more greater than the mature follicle, in the ovary for a long period of 10 days or more. According to Hanwoo (Korean Native Cattle) Disease Management, about 70% of Korean cattle reproductive disorders are caused by ovarian cysts, and mainly occur for 1 to 4 months after delivery.

There are three types of ovarian cysts such as a follicular cyst (FC), a luteal cyst (LC), and a cystic corpora lutea (CL) of the ovary. Thereamong, the follicular cyst (FC) is the most common disease causing failure of ovulation and luteal formation.

This is the occurrence of abnormal hormone secretion due to pregnancy and delivery, and as a typical symptom, nymphomania which is frequent estrus, and non-estrus appear in a ratio of about 80:20. In the case in which the estrus period is different from the usual cycle due to an ovarian cyst, it may be easy to miss the breeding period, and an increase in the breeding interval and fertilization frequency is a cause of lowering productivity of cattle.

To accurately detect an ovarian cyst, it is necessary to check whether the size of the ovary is 2.5 cm or more through rectal ultrasound tests, but such tests increase the costs and it may be practically difficult to periodically test all animal subjects.

Biomarkers may be collected by using the biosensor capsule 200, which is a bio-insertable sensor according to an example embodiment of the present disclosure, and ovarian cysts may be determined through an analysis of the collected biomarkers through artificial intelligence (AI).

Artificial intelligence refers to the field of researching into artificial intelligence or a methodology that may create the AI, and machine learning refers to a field that defines various problems dealt with in the field of artificial intelligence and studies methodologies to solve the problem. Machine learning may also be defined as an algorithm that improves the performance of a task through continuous experience.

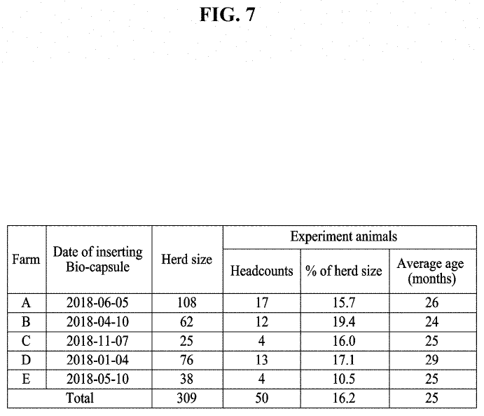

FIG. 7 illustrates information on experimental subjects, and experimental animals were selected from five Korean-cattle breeding cattle farms in which data was accumulated for a predetermined period of time or longer after administration of biocapsules. Among the animals for that sufficient information to analyze each animal characteristic was secured by accumulating data before the experiment, 50 animals that completed delivery in December 2018 were selected as experimental subjects.

The periods of estrus after recursive estrus, for each animal during the experiment period (1 Jan. 2019-30 Apr. 2019) were compared and observed. The recursive estrus refers to first estrus that occurs after delivery and the end of a lactation phase.

Experimental animals were selected at a rate of 10 to 20% of the total subjects for each farm, and as of the start date of the experiment (Jan. 1, 2019), all subjects were 24 months or more of age and have completed delivery. In detail, since ovarian cysts appear in parous animals (with experience of giving birth), the parous animal subjects were selected and tested.

Biomarkers of cattle were measured using the biosensor capsule 200. A temperature sensor and a 3-axis acceleration sensor mounted in the biosensor capsule measured a temperature change and activity of the cattle at 10 minute intervals, respectively. When the temperature of cattle is measured with the temperature sensor included in the biosensor capsule 200, bioinformation may be collected without external influence. According to example embodiments, without limiting to the 3-axis acceleration sensor, the activity may also be measured with a 6-axis acceleration sensor or the like.

The measurement period may be set longer than that described above, and as the period is shortened, detecting estrus may be facilitated, but the measurement period may be adjusted in consideration of the battery consumption of the biosensor capsule 200.

The deep temperature of cattle may be measured finely in units of 0.1° C. In the case of temperature information, a measured value may be used as is in monitoring a change, but a difference from an average value may be represented in a normalized graph using the average and standard deviation.

Behavioral characteristics such as mounting of cattle, increased gait of cattle, lethargy of cattle and the like may be analyzed based on a vector value synthesizing three-axis acceleration values. For example, the activity may be measured by Equation 1 below.

v=\sqrt{X²+Y²+Z²}.[Equation 1]

In some cases, the activity of an animal subject may be relatively greatly measured due to the behavioral aspects of livestock such as cattle's excessive walking or lame cows. Acceleration data of several axes may be used to distinguish activity information due to the movement of animal from activity information due to animal's behavior such as mounting. With the activity information, the presence or absence of estrus may be detected by patterning the direction of movement according to a specific action, for example, mounting, that appears during estrus.

The temperature information and activity information obtained from the biosensor capsule 200 may be analyzed using an AI algorithm. An artificial neural network (ANN) is a model used in machine learning, and refers to an overall model having problem-solving capabilities, which is composed of artificial neurons (nodes) that form a network by combining synapses. The artificial neural network may be defined by a connection pattern between neurons of different layers, a learning process for updating model parameters, and an activation function for generating an output value.

The artificial neural network may include an input layer and an output layer, and may selectively include a plurality of hidden layers. Each layer includes a plurality of neurons, and the artificial neural network may include synapses connecting neurons and neurons. In the artificial neural network, each neuron may output a function value of an activation function for input signals, weights and biases, input through synapses.

Model parameters refer to parameters determined through learning, and include weights of synaptic connections, biases of neurons, and the like. In addition, hyperparameter refers to a parameter that should be set before learning in a machine learning algorithm, and includes a learning rate, iteration count, mini-batch size, initialization function, and the like.

The purpose of learning artificial neural networks may be regarded as determining model parameters that significantly reduce a loss function. The loss function may be used as an index to determine an optimal model parameter in the learning process of the artificial neural network.

Machine learning may be classified into supervised learning, unsupervised learning, and reinforcement learning according to the learning method.

Supervised learning refers to a method of learning an artificial neural network in a state in which a label for training data is given, and the label may indicate a correct answer or a result value that the artificial neural network should infer when the training data is input to the artificial neural network. Unsupervised learning refers to a method of learning an artificial neural network in a state in which a label for training data is not given. Reinforcement learning may indicate a learning method in which an agent defined in a certain environment learns to select an action or action sequence that maximizes a cumulative reward in each state.

Among artificial neural networks, machine learning implemented as a deep neural network (DNN) including a plurality of hidden layers is also referred to as deep learning, and the deep learning is a part of machine learning. Hereinafter, machine learning is used in a sense including deep learning.

A machine learning system provided by combining Fully Convolutional Networks (FCN) that is a Semantic Segmentation Algorithm Model and a Long Short-term Memory (LSTM) that is used for time-series data linkage analysis as an application of a Recurrent Neural Network, among AI application techniques, is used to perform multi-class classification.

The LSTM technique is a learning method for processing time series data like a Recurrent Neural Network (RNN) technique, and may record input information longer than the RNN technique, to allow learning of relatively long data to learn.

The RNN technique is a technique for calculating prediction data by using previous information as input data for immediately subsequent information. However, if the data is too long such that the distance between the previous information and current information increases, a backpropagation gradient gradually decreases, resulting in a problem in which the learning ability greatly deteriorates. The LSTM is a technique designed to prevent this problem. The LSTM is a structure in which a cell-state is added to a hidden state of the RNN, and may be comprised of processes of updating and outputting information to be discarded from the input cell state, information to be input to a long term layer, and existing information.

The FCN technique is a method of obtaining improved data by convolution multiplication of a filter with raw data. Through the FCN technique, noise of raw data may be reduced, and thus more reliable prediction values may be obtained.

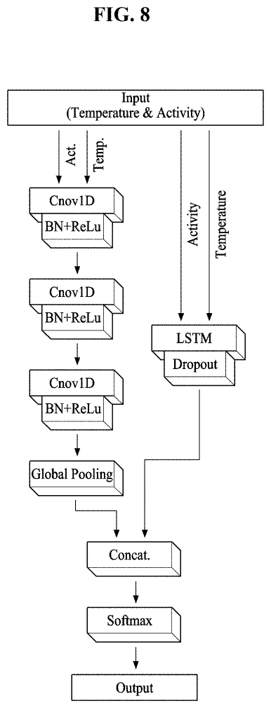

FIG. 8 is a schematic diagram illustrating an algorithm for predicting ovarian cyst by putting temperature information and activity information in an LSTM-FCN model.

In the LSTM-FCN model, the type of biomarker change is normalized in a predetermined range from 0 to 1 is classified as estrus and an ovarian cyst symptom of an animal. With the probability of an ovarian cyst, whether or not an ovarian cyst actually occurs may be determined by performing machine learning training and testing in the Microsoft Azure Machine Learning Tool, based on data of about 500 million cattle.

In detail, noise appearing in collected data due to a difference of each animal may be compensated by grasping the cycle of estrus of each animal through temperature information and activity information before pregnancy and delivery of an experimental animal and by reflecting the difference.

In more detail, as illustrated in FIG. 8, the input time series information of the system is comprised of temperature information and activity information. The temperature information may be normalized by expressing a difference from an average value using a mean and standard deviation.

The normalized temperature information and activity information are input to the Long short-term memory (LSTM) and one-dimensional convolution layer according to each time frame. With respect to a probability value, for example, a probability of an ovarian cyst state and a probability of a normal state, an output value are calculated by connection and processing through a softmax function. In the case in which the cow is determined to be in an ovarian cyst state, as illustrated in [Equation 2] below, the output value is [100, 0] as a percentage, and in the case of a normal state, the output value is [0, 100].

Probability of ovarian cyst state=100−Probability of normal state. [Equation 2]

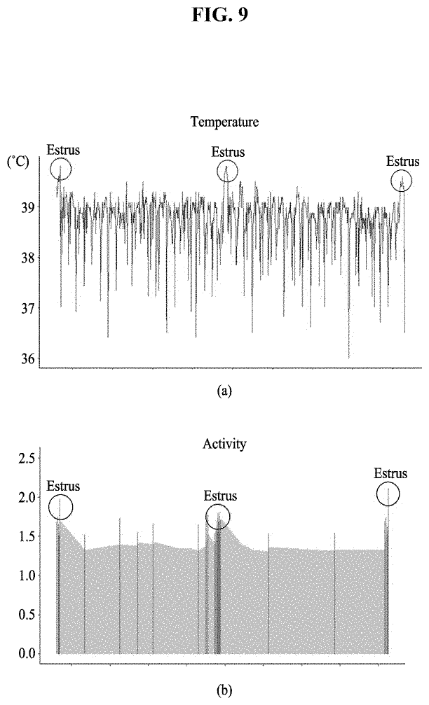

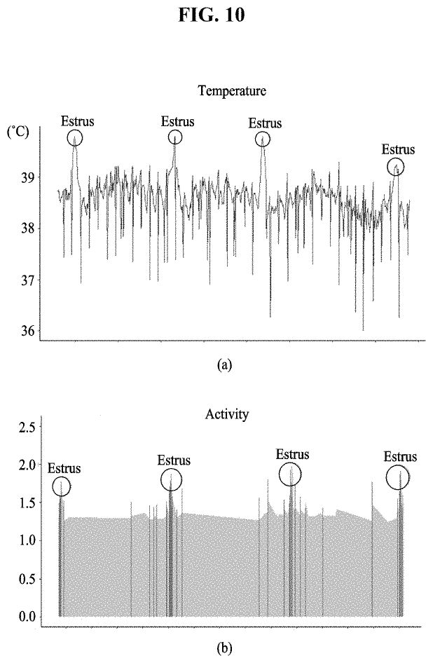

(a) and (b) in FIG. 9 are graphs illustrating temperature information and activity information of an animal in a normal state, and (a) and (b) in FIG. 10 are graphs illustrating temperature information and activity information of an animal in an ovarian cyst state.

The graphs illustrate data for about two months from a date of recursive estrus, and the shaded portions of the activity information indicate an average value of activity information for 1 hour. The graphs illustrate a form in which a vertical line is drawn and the activity is rapidly increased when there is a sudden movement.

In the case of an animal in the normal state in FIG. 9, estrus was detected three times for two months, including recursive estrus, and in the case of an animal in the ovarian cyst state in FIG. 10, estrus was detected four times for two months, including recursive estrus. Compared to the normal pattern, frequent estrus is detected, and it may be determined as a behavior caused by an ovarian cyst.

Referring to FIGS. 9 to 10, when the temperature information and the activity information of the livestock exceed each threshold, it may be determined that a singular point has been expressed.

Although FIGS. 9 to 10 illustrate the example embodiment using only temperature information and activity information, in case of using other indicators, for example, electrical conductivity, when each piece of information exceeds a threshold, it may be determined that a singular point has been expressed.

When at least one of data collected by a plurality of sensors exceeds the threshold, it may be determined that the singular point is expressed.

The presence or absence of estrus may be determined, based on an expression time and duration of a first singular point in which the temperature information exceeds a temperature threshold and a second singular point in which the activity information exceeds an activity threshold.

When the first singular point and the second singular point appear at the same time, and a first duration, for example, 1 to 2 hours, lasts, it may be determined as estrus. In general, since estrus lasts about 18 hours, in the case in which the first singular point and the second singular point are expressed for a relatively short period of time, for example, 20 to 30 minutes, it may not be determined as estrus.

Alternatively, in the case in which only one of the first singular point and the second singular point is expressed, when the expression of the singular point continues a second duration or more, this case may also be determined as estrus. In the case of the second duration, the second duration for the first singular point and the second duration for the second singular point may be different from each other. The second duration may be set to be, for example, 6 hours or more, longer than the first duration that is a duration when the first and second singular points are simultaneously expressed.

A normal estrus period is derived based on previously analyzed population data, and when estrus is expressed in a period different from the normal estrus period, it may be determined as abnormal estrus. For example, in general, since cows show estrus for about 21 days, when cows have an estrus period of less than 19 days or more than 23 days, it may be determined as abnormal estrus.

The controller 330 of the disease management server 300 may determine estrus using temperature information and activity information, and when the difference from previous information is relatively great by using the temperature information and the activity information collected before delivery for each animal, it may be determined as abnormal estrus, which does not match a normal estrus cycle.

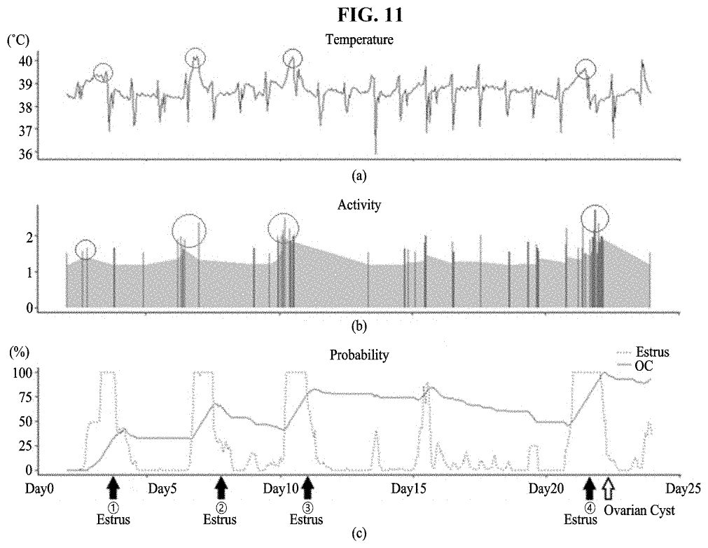

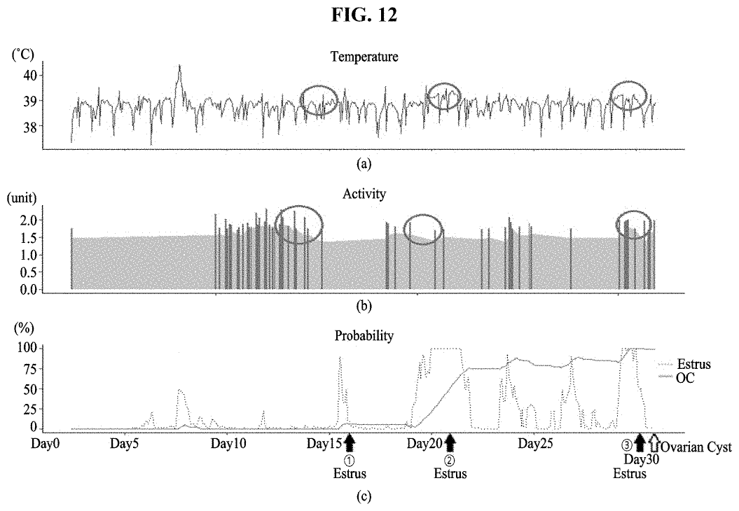

(a) in FIG. 11 and (a) in FIG. 12 are a temperature graph of an animal exhibiting a pattern of an ovarian cyst, and (b) in FIG. 11 and (b) in FIG. 12 are an activity graph of an animal exhibiting a pattern of an ovarian cyst. (c) in FIG. 11 and (c) in FIG. 12 are graphs illustrating a probability of estrus and a probability of ovarian cyst, based on temperature information and activity information, for example, analyzed by the controller 330 of the disease management server 300 of FIG. 3 using an AI algorithm.

The graphs are illustrated based on temperature information and activity information measured from about 7 weeks (42 days) after delivery, and points determined as the time of estrus are indicated by arrows and circled numbers.

Circled portions of the temperature graphs of (a) in FIG. 11 and (a) in FIG. 12 and the activity graphs of (b) in FIG. 11 and (b) in FIG. 12 represent the first and second singular points exceeding a temperature critical point and an activity critical point. (c) in FIG. 11 and (c) in FIG. 12 are graphs illustrating a probability of estrus (indicated by dotted line) and a probability of ovarian cyst (indicated by solid line), and the arrows indicate the parts determined to be estrus.

When the first singular point and the second singular point occur simultaneously, it may be determined as estrus, and the points determined to be estrus are indicated by arrows in (c) in FIG. 11 and (c) in FIG. 12. When the first singular point and the second singular point appear at the same time, the probability of estrus increases, and when the singular points last for a predetermined period of time, e.g., 1 hour or more, it may be determined as estrus.

When the temperature and activity decrease, the estrus ends and the probability of estrus decreases. When estrus is expressed, a probability of occurrence of animal's ovarian cyst may increase. In particular, as the frequency of estrus is shorter, the probability of ovarian cyst may significantly increase.

When the first estrus (recursive estrus) ((D) appears early after delivery as illustrated in (c) in FIG. 11, the probability of ovarian cyst is increased, whereas when recursive estrus ((D) is late as illustrated in (c) in FIG. 12, the probability of ovarian cyst may gentle increase during recursive estrus.

As time elapses after estrus, the probability of ovarian cyst slowly decreases, but in the case in which estrus is detected again, the probability of ovarian cyst may increase again. In the case in which only one of the first singular point and the second singular point appears, it may not be determined as estrus expression, but it is determined as an abnormal symptom and the probability of ovarian cyst may be increased in which the probability is a lower level than that in the case determined as estrus.

As illustrated in (c) FIG. 11, when four or more times of estrus including recursive estrus are detected for around 21 days which is one cycle, the probability of ovarian cyst is 100 at the fourth estrus, and an alarm for suspicion of ovarian cyst may be provided to the user device 400.

Alternatively, as illustrated in (c) in FIG. 12, when three or more estrus is detected including recursive estrus for a short period of time, it is also determined as an ovarian cyst, and an alarm may be provided to the user device 400.

The probability of ovarian cysts is not determined simply by the number of estrus onset, but the extent of an increase in ovarian cyst probability may vary depending on each estrus period length, irregularity in estrus cycle, intensity of estrus, duration of estrus, or the like.

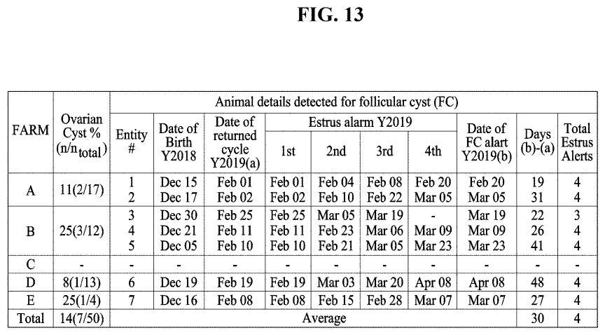

In this way, presence or absence of estrus is determined based on temperature information and activity information. FIG. 13 is a table illustrating the results of determining whether or not an ovarian cyst for each animal subject is expressed by changing the ovarian cyst probability depending on temperature information, activity information, and presence or absence of estrus.

Of the 50 experimental animals, 7 animals were determined as ovarian cysts, and two animals from Farm A and three animals from Farm B were found as ovarian cysts. There was no ovarian cyst found in Farm C. One each was found in Farm D and Farm E. The time point to be determined as an ovarian cyst is 19 to 48 days from the date of recursive estrus, and is usually expressed between a first estrus period (21 days) and a second estrus period (42 days) from the date of recursive estrus, except for maximum and minimum values.

Although there is a difference between respective experimental subjects, estrus was expressed four times for an average of 30 days, which entered estrus every about 10 days, and it can be seen that about a week and a half is earlier than a normal cycle.

By predicting the presence or absence of an ovarian cyst through biometric information detected through the biosensor capsule 200 and extracting an animal subject to be examined by a veterinarian, the waste of manpower for unnecessary examination may be reduced. In this experiment, precise examination was carried out on only 7 out of 50 animals.

In order not to omit an ovarian cyst animal, omission of an animal with an ovarian cyst may also be prevented by increasing the rate of increase in the probability of ovarian cyst even for small changes.

However, in this case, since the number of unnecessary ovarian cyst tests increases, the probability of ovarian cyst according to changes in temperature and activity and the presence or absence of estrus may be adjusted through artificial intelligence (AI) analysis using existing information. Using the information collected from the biosensor capsule 200 according to an example embodiment of the present disclosure, the disease management server 300 quickly detects an animal suspected of having a reproductive disorder (ovarian cyst), and may provide intensive care (veterinarian examination and hormone prescription) to the animal, thereby increasing farm productivity.

The units described herein may be implemented using hardware components and software components. For example, the hardware components may include microphones, amplifiers, band-pass filters, audio to digital convertors, non-transitory computer memory and processing devices. A processing device may be implemented using one or more general-purpose or special purpose computers, such as, for example, a processor, a controller and an arithmetic logic unit (ALU), a digital signal processor, a microcomputer, a field programmable gate array (FPGA), a programmable logic unit (PLU), a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a processing device may include multiple processors or a processor and a controller. In addition, different processing configurations are possible, such a parallel processors.

The software may include a computer program, a piece of code, an instruction, or some combination thereof, to independently or collectively instruct or configure the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network coupled computer systems so that the software is stored and executed in a distributed fashion. The software and data may be stored by one or more non-transitory computer readable recording mediums. The non-transitory computer readable recording medium may include any data storage device that can store data which can be thereafter read by a computer system or processing device.

Example embodiments include non-transitory computer-readable media including program instructions to implement various operations embodied by a computer. The media may also include, alone or in combination with the program instructions, data files, data structures, tables, and the like. The media and program instructions may be those specially designed and constructed for the purposes of example embodiments, or they may be of the kind well known and available to those having skill in the computer software arts. Examples of non-transitory computer-readable media include magnetic media such as hard disks, floppy disks, and magnetic tape; optical media such as CD ROM disks; magneto-optical media such as floptical disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter. The described hardware devices may be configured to act as one or more software modules in order to perform the operations of the above-described example embodiments, or vice versa.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents.

Therefore, the scope of the disclosure is defined not by the detailed description, but by the claims and their equivalents, and all variations within the scope of the claims and their equivalents are to be construed as being included in the disclosure.

1. A method of managing livestock disease comprising:
receiving biometric information of livestock from a biosensor capsule settled in a body of the livestock;
detecting a singular point deviating from a reference value, based on the biometric information;
monitoring a health state by increasing a disease probability of the livestock when the singular point is detected; and
detecting a disease by determining an abnormality in the health state of the livestock when the disease probability of the livestock is a first threshold value or more.

2. The method of claim 1, wherein the biometric information comprises temperature information of the livestock and activity information of the livestock.

3. The method of claim 2, wherein the activity information of the livestock comprises at least one of angular velocity information and acceleration information sensed by the biosensor capsule.

4. The method of claim 2, wherein the detecting of the singular point comprises,
an estrus detection operation of determining estrus when at least one of a first singular point in which the temperature information exceeds a temperature threshold value or a second singular point in which the activity information exceeds an activity threshold value is expressed,
wherein when the estrus is detected, a range of an increase in the disease probability of the livestock is greater.

5. The method of claim 4, wherein in the estrus detection operation, when a duration of the first singular point or a duration of the second singular point lasts a first duration or more, it is determined as the estrus.

6. The method of claim 4, wherein in the estrus detection operation, when both the first singular point and the second singular point are expressed, it is determined as the estrus.

7. The method of claim 4, wherein in the estrus detection operation, when only one of the first singular point and the second singular point is expressed and a duration of an expressed singular point lasts a second duration or more, it is determined as the estrus.

8. The method of claim 4, wherein in the monitoring of the health state, a range of change in the disease probability of the livestock is determined according to a frequency of expression of the estrus, an irregularity of expression of the estrus, a duration of expression of the estrus, and intensity of expression of the estrus.

9. The method of claim 8, wherein as the frequency of expression of the estrus increases, the expression of the estrus is irregular and the duration of the estrus increases, a range of an increase in the disease probability of the livestock is greater.

10. The method of claim 4, wherein in the monitoring of the health state, when the estrus detection operation is expressed three times or four times or more within a second estrus period, the disease probability of the livestock is controlled to be equal to or higher than the first threshold value.

11. The method of claim 2, wherein in the monitoring of the health state, after the detecting of the singular point and before detection of a next singular point, the disease probability of the livestock is lowered.

12. The method of claim 1, further comprising an alarm operation of notifying a user of a disease of the livestock when the disease probability is equal to or higher than a set value.

13. The method of claim 1, wherein the abnormality in the health state of the livestock is an ovarian cyst.

14. The method of claim 1, further comprising inserting and seating the biosensor capsule in a stomach or a vagina of the livestock.

15. A disease management server managing a disease of livestock, comprising:
a communication module receiving biometric information of the livestock from a biosensor capsule settled in a body of the livestock; and
a controller analyzing the biometric information, determining a health state of the livestock based on an analysis result, and transmitting information on the health state of the livestock to a user managing the livestock,
wherein the controller is configured to,
detect a singular point deviating from a reference value based on the biometric information,
increase a disease probability of the livestock when the singular point is detected, and
transmit an abnormality in the health state of the livestock to the user when the disease probability of the livestock is equal to or higher than a first threshold value.

16. The disease management server of claim 15, wherein the biometric information comprises temperature information of the livestock and activity information of the livestock, and
wherein the controller is further configured to:
determine estrus when at least one of a first singular point in which the temperature information exceeds a temperature threshold value or a second singular point in which the activity information exceeds an activity threshold value is expressed, and
increase the disease probability of the livestock.

17. The disease management server of claim 16, wherein the controller is configured to determine a range of a change in the disease probability of the livestock depending on a frequency of expression of the estrus, irregularity of expression of the estrus, a duration of expression of the estrus, and intensity of expression of the estrus.

18. The disease management server of claim 17, wherein the controller is configured to control the disease probability of the livestock to be equal to or higher than the first threshold value when the estrus is expressed three times or four times or more within a second estrus period.

19. The disease management server of claim 15, wherein the abnormality in the health state of the livestock is an ovarian cyst.
